(19) United States
(12) Patent Application Publication
(10) Pub. No.: US 2018/0126016 A1
(43) Pub. Date: May 10, 2018

(54) Catalyst Unit for Splitting a Decontamination Agent Introduced Into a Containment for a Decontamination Process

(71) Applicant: Skan AG, Allschwil (CH)

(72) Inventors: Scheuber Olivera, Schönenbuch (CH); Volker Sigwarth, Sisseln (CH)

(73) Assignee: Skan AG, Allschwil (CH)

(21) Appl. No.: 15/566,938

(22) PCT Filed: Apr. 11, 2016
(86) PCT No.: PCT/CH2016/000062

§ 371 Date: Oct. 16, 2017

(30) Foreign Application Priority Data
Apr. 16, 2015 (EP) 15405030.6

(51) Int. Cl. A61L 2/00; A61L 2/20; B01D 53/00; B01D 53/86; B01J 21/04; B01J 21/18; B01J 23/50; B01J 35/00; B01J 35/10; B01J 37/00

(52) U.S. Cl. CPC A61L 2/0094; B01D 2259/804; B01D 53/007; B01D 53/8671; B01J 21/04; B01J 21/18; B01J 23/50; B01J 35/0013; B01J 35/004; B01J 35/1066; B01J 37/0009; B01D 2255/104; B01D 2255/904; B01D 2259/4533; A61L 2/208

(57) ABSTRACT

The catalyst unit (1) for splitting a decontamination agent introduced into a containment for a decontamination process has at least one catalyst element (21). This catalyst element (21) first consists of a carrier material made of aluminum ceramics or activated carbon, and also of a catalytically active component in the form of nanoparticles, applied to the carrier material by means of chemical plating, and made of silver or silver oxide or of a silver and silver oxide mixture. The catalytically active component on the at least one catalyst element (21) is present in the range from 0.05 weight percent to 0.5 weight percent relative to the carrier material. The at least one catalyst element (21) has a catalytically effective surface in the range of up to 320 m² per gram of used material, as a combination of carrier material plus applied catalytically active component. With the catalyst unit (1), an non-critical concentration of non-degraded decontamination agent of lower than 0.5 ppm, preferentially a maximum of 0.1 ppm can be achieved. The containment is configured in particular as an isolator, sluice or RABS (Restricted

Access Barrier System), as well as being of a mobile and stationary type, such as means for transportation, and spaces for treatment, isolation and/or diagnosis of patients, as well as production spaces and laboratories.

### FIELD OF THE INVENTION

The present invention relates to a catalytic unit for splitting a decontamination agent introduced into a containment for the purposes of a decontamination process. The catalytic unit possesses at least one catalytic element. The individual catalytic element is provided a catalytically active component applied to a carrier material. Containments are in particular isolators, for example for the pharmaceutical-chemical industry, locks and safety work stations, for example for microbiological operations or operations involving toxic entities. The term further comprehends all types of restricted access barrier systems (RABSs), including mobile and stationary kinds, such as means of transport and rooms for treatment, isolation and/or diagnosis of patients, and also production rooms and laboratories.

### PRIOR ART

DE 197 36 336 A1 discloses a catalytic unit for decomposition of air pollutants, especially hydrogen peroxide. The proposal is for a defined depth of a loose bed of particles having a specific particle size. The loose bed consists essentially of purely metal oxide of the metals Cu, Mn, Fe, Co, Ni or mixtures thereof, wherein the metal oxides may be on a carrier material. The metal oxide or the metal oxide mixture may comprise admixtures of noble metals, such as Pt or Pd. However, this catalytic unit requires a relatively large number of catalytic plates and/or a corresponding thickness, so a relatively high pressure drop occurs in the system and therefore the deployment inside containments causes comparatively high costs or the efficiency remains rather modest. In addition, such a catalytic unit is burdensome and costly to manufacture, explaining why its commercial use is rather limited in relation to the potential benefit.

EP 0 882 492 A1 relates to apparatus for ridding a gas stream of entrained hydrogen peroxide, again by using a catalytic unit. The catalytically effective material is manganese oxide, iron oxide or copper oxide, applied to a hydrophobic carrier material, for example a zeolite in the form of pourable pieces. The catalytic unit proposed therein likewise requires a considerable size, meaning a high pressure drop in operation and therefore higher costs being caused by deployment inside containments or that altogether the efficiency should be higher. The manufacturing process and its attendant costs also appear to be disadvantageous.

Finally, WO 2011/085 735 A1 contains a decontamination arrangement, especially for pharmatechnical applications, having a room to be decontaminated, especially an isolator room, and also having a purifying device for removing gaseous and/or vaporous decontamination agent from the room air. The decontamination agent is specifically hydrogen peroxide. The room air is circulated by at least one blower through the purifying device, which includes a catalytic unit which, for chemically splitting the decontamination agent, is based on a manganese oxide or palladium coated carrier structure as reactive or catalytically effective material. The carrier structure is configured as an open cell metal foam structure or as a lattice structure whereto the effective material has been applied electrolytically. This catalytic unit is likewise judged to be in need of improved efficiency and also as too costly to manufacture.

### OBJECT OF THE INVENTION

In view of the prior art catalytic units offering rather modest efficiencies while being relatively expensive to manufacture, their use for splitting decontamination agents is only sensible to a limited extent. The invention therefore has for its object to create a more efficient catalytic unit whereby nearly 100% degradation is achieved for decontamination agent present in an essentially gaseous process stream.

The improved efficiency sought includes the objective that the catalytic unit be capable of processing even large volume flows at very low pressure drops. The catalytic unit to be created shall ensure that the water fractions occurring in the decontamination process are brought to an advantageous level. The process employed for manufacturing the catalytic unit and also the materials used for manufacturing shall further ideally be inexpensive.

One specific object for the catalytic unit to be created consists in the decontamination agent to be split occurring in the process stream of a performed decontamination in a containment as per the broad definition in the technical field.

### SUMMARY OF THE INVENTION

The proposed catalytic unit for splitting a decontamination agent introduced into a containment for the purposes of a decontamination process possesses at least one catalytic element. This catalytic element consists initially of a carrier material formed from aluminum ceramic or activated carbon and further of a catalytically active component applied to the carrier material by chemical plating and taking the form of nanoparticles formed from silver or silver oxide or a mixture of silver and silver oxide.

Specific embodiments of the invention will now be defined: The catalytically active component comprises from 0.05 weight percent to 0.5 weight percent, preferably at 0.1 weight percent, of the carrier material in the at least one catalytic element. The at least one catalytic element has a catalytically effective surface area ranging up to 320 m² per gram of catalyst material used, being the combination of carrier material plus applied catalytically active component. The specific surface area [m² per gram] of the catalyst material used, being the combination of carrier material plus applied catalytically active component, is not decreased by the applied nanoparticles in relation to the specific surface area [m² per gram] of the carrier material alone, before application of the nanoparticles.

The catalytic unit comprises:
a) at least one plate-shaped catalytic element or two or more consecutively packed such catalytic elements having a gastight covering possessing an open inlet for inflowing a process stream into the catalytic unit and an open outlet for outflowing the process stream out of the catalytic unit; or
b) at least one hollow cylindrical catalytic element or two or more concentrically packed such catalytic elements having a gastight covering possessing an open inlet for inflowing the process stream into the catalytic unit and an open outlet for outflowing the process stream out of the catalytic unit.

The catalytic unit is for splitting a decontamination agent which:
a) has a sporicidal effect which causes at least a 3-log reduction;
b) preferably passes in aerosol form into the containment; and
c) preferably is hydrogen peroxide [H₂O₂] or nitrogen dioxide [NO₂] or peroxyacetic acid [C₂H₄O₃] or a mixture of hydrogen peroxide [H₂O₂] and peroxyacetic acid [C₂H₄O₃].

The containment is specifically configured as isolator, lock or restricted access barrier system (RABS), including mobile and stationary kinds, such as means of transport and rooms for treatment, isolation and/or diagnosis of patients, and also production rooms and laboratories.

The catalytic unit is for splitting a decontamination agent present for a decontamination process in a gas stream, passing through the catalytic unit and exiting into an area or a second containment or into the open atmosphere. Here the exiting gaseous medium has an uncritical concentration of non-degraded decontamination agent amounting to less than 0.5 ppm, whereas a concentration above 1.0 ppm is defined as critical. Preferably the attained uncritical concentration of non-degraded decontamination agent is not more than 0.1 ppm.

At least one activated UV light source is directed at the at least one catalytic element during the splitting process for the purpose of intensifying the catalytic effect.

The at least one UV light source is disposed:
a) outside the catalytic unit; or
b) between adjacent catalytic elements; or
c) centrally in a hollow cylindrical catalytic unit comprising one or more catalytic elements.

Alternatively, one UV light source is arranged in each case:
a) outside the catalytic unit; and
b) in each case between adjacent catalytic elements; and
c) centrally in a hollow cylindrical catalytic unit comprising one or more catalytic elements.

To maximize the intensification of the catalytic effect:
a) the at least one UV light source is constituted and positioned and/or guide elements are disposed on the UV light source and/or on the catalytic unit such that a maximum of the radiative effect of the UV light source is attainable on a maximum of the surface area of the one catalytic element or the two or more catalytic elements; and/or
b) the wavelength of the at least one UV light source is adjusted according to the constitution of the carrier influenced catalytically active component.

### BRIEF DESCRIPTION OF THE ATTACHED DRAWINGS

In the drawings:

FIG. 1A—shows a catalytic unit, illustratively consisting of a single catalytic element in plate shape, as a block diagram;

FIG. 1B—shows the catalytic unit of FIG. 1A, having a UV radiation source disposed either side of the catalytic unit on the outside thereof, as a block diagram;

FIG. 2A—shows a catalytic unit consisting of a single catalytic element in the form of a concentric hollow cylinder having one UV radiation source disposed outside and in the center of the catalytic unit, as a block diagram;

FIG. 2B—shows the catalytic unit of FIG. 2A with interchanged inlet and outlet, as a block diagram; and

FIG. 2C—shows the catalytic unit of FIG. 2A, in vertical section along the line A-A.

### EXEMPLARY EMBODIMENT

Referring to the accompanying drawings, the detailed description follows of the catalytic unit the invention provides for splitting a decontamination agent present in an at least essentially gaseous process stream. This is done by setting forth two basic versions and mentioning various modifications thereto.

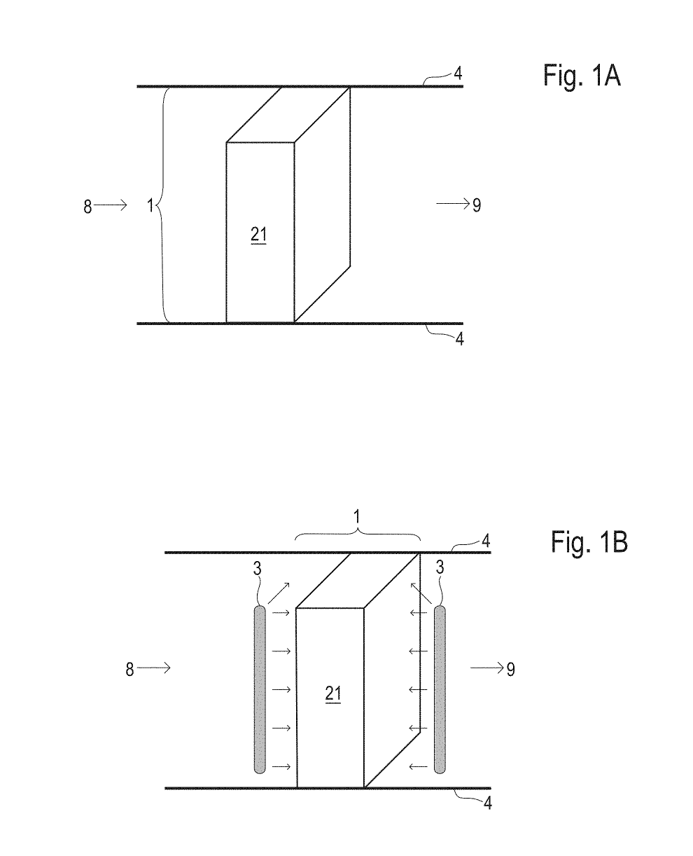

### FIG. 1A

This first version catalytic unit 1 consists, illustratively, of a single catalytic element 21 in plate form and is provided a gastight covering 4 possessing an open inlet 8 for inflowing a process stream into the catalytic unit 1 and an open outlet 9 for outflowing the process stream out of the catalytic unit 1. This leads the entire process stream from the inlet 8 through the catalytic unit 1 to the outlet 9. Alternatively, the catalytic unit 1 may also consist of two or more consecutively packed such plate-shaped catalytic elements 21.

The at least one catalytic element 21 consists of:
a) a carrier material formed from aluminum ceramic or activated carbon; and
b) a catalytically active component applied to the carrier material by chemical plating and taking the form of nanoparticles formed from silver or silver oxide or a mixture of silver and silver oxide.

The catalytically active component comprises from 0.05 weight percent to 0.5 weight percent, preferably at 0.1 weight percent, of the carrier material in the at least one catalytic element 21. This at least one catalytic element 21 has a catalytically effective surface area ranging up to 320 m² per gram of catalyst material used, being the combination of carrier material plus applied catalytically active component.

The specific surface area [m² per gram] of the catalyst material used, being the combination of carrier material plus applied catalytically active component, is not decreased by the applied nanoparticles in relation to the specific surface area [m² per gram] of the carrier material alone, before application of the nanoparticles.

The catalytic unit 1 is for splitting a decontamination agent which:
a) is present for a decontamination process in a gas stream, passing through the catalytic unit 1 and exiting into an area or a second containment or into the open atmosphere, wherein the exiting gaseous medium has an uncritical concentration of non-degraded decontamination agent amounting to less than 0.5 ppm, whereas a concentration above 1.0 ppm is defined as critical, and preferably the attained uncritical concentration of non-degraded decontamination agent is not more than 0.1 ppm;
b) has a sporicidal effect which causes at least a 3-log reduction;
c) preferably passes in aerosol form into the containment; and
d) preferably is hydrogen peroxide [H₂O₂] or nitrogen dioxide [NO₂] or peroxyacetic acid [C₂H₄O₃] or a mixture of hydrogen peroxide [H₂O₂] and peroxyacetic acid [C₂H₄O₃].

The containment is specifically configured as isolator, lock or restricted access barrier system (RABS), including mobile and stationary kinds, such as means of transport and rooms for treatment, isolation and/or diagnosis of patients, and also production rooms and laboratories.

### FIG. 1B

At least one activated UV light source 3 is directed at the at least one catalytic element 21 during the splitting process for the purpose of intensifying the catalytic effect; here a UV light source 3 is disposed either side of the catalytic unit 1 comprising just the one catalytic element 21. Where the catalytic unit 1 consists of two or more identical mutually spaced apart catalytic elements 21, UV light sources 3 may alternatively or additionally also be provided between adjacent catalytic elements 21.

To maximize the intensification of the catalytic effect:
a) the at least one UV light source 3 is and/or the UV light sources 3 positioned on both sides are constituted and positioned and/or guide elements are disposed on the UV light source 3 and/or on the catalytic unit 1 such that a maximum of the radiative effect of the UV light sources 3 is attainable on a maximum of the surface area of the one catalytic element 21 and/or all of the catalytic elements 21 in the catalytic unit 1; and/or
b) the wavelength of the at least one UV light source 3 is adjusted according to the constitution of the carrier influenced catalytically active component.

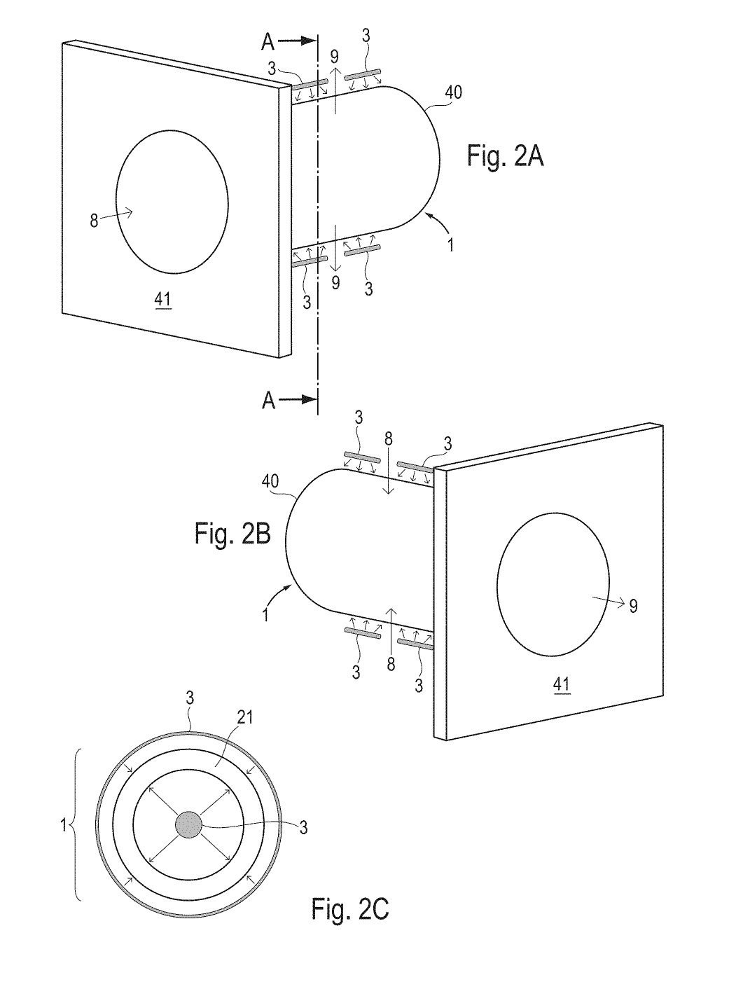

### FIGS. 2A to 2C

The second version catalytic unit 1 consists most simply of a single catalytic element 21, albeit now in the form of a hollow cylinder. The drawn depiction of a catalytic unit 1 having more than one hollow cylindrical catalytic element 21 has been eschewed, nonetheless such an expanded construction is realizable analogously to the alternative referred to in connection with FIG. 1A. However, catalytic unit 1 is similarly provided the optional UV light sources 3, to the max with one UV radiation source 3 disposed both outside and in the center of catalytic unit 1, as apparent in FIG. 2C.

The cylindrical shaped catalytic unit 1 has a gastight cap 40 at one axial end and a plate-shaped, for example, assembly element 41 at the other end. In one modification, the assembly element 41 is situated on the side of inlet 8, and in that case the outlet 9 is formed by the shell surface of catalytic unit 1 (see FIG. 2A).

In the other modification, the assembly element 41 is disposed on the side of outlet 9, and in that case the inlet 8 is effected via the shell surface of catalytic unit 1 (see FIG. 2B).

1. A catalytic unit for splitting a decontamination agent introduced into a containment for the purposes of a decontamination process, wherein
the catalytic unit possesses at least one catalytic element consisting of
a carrier material formed from aluminum ceramic or activated carbon; and
a catalytically active component applied to the carrier material by chemical plating and taking the form of nanoparticles formed from silver or silver oxide or a mixture of silver and silver oxide.

2. The catalytic unit as claimed in claim 1, wherein
the catalytically active component comprises from 0.05 weight percent to 0.5 weight percent, of the carrier material in the at least one catalytic element;
the at least one catalytic element has a catalytically effective surface area ranging up to 320 m² per gram of catalyst material used, being the combination of carrier material plus applied catalytically active component; and
the specific surface area [m² per gram] of the catalyst material used, being the combination of carrier material plus applied catalytically active component, is not decreased by the applied nanoparticles in relation to the specific surface area [m² per gram] of the carrier material alone, before application of the nanoparticles.

3. The catalytic unit as claimed in claim 1, wherein the catalytic unit comprises:
at least one plate-shaped catalytic element or two or more consecutively packed such catalytic elements having a gastight covering possessing an open inlet for inflowing a process stream into the catalytic unit and an open outlet for outflowing the process stream out of the catalytic unit; or
at least one hollow cylindrical catalytic element or two or more concentrically packed such catalytic elements having a gastight covering possessing an open inlet for inflowing the process stream into the catalytic unit and an open outlet for outflowing the process stream out of the catalytic unit.

4. The catalytic unit as claimed in claim 1, wherein
the catalytic unit is for splitting a decontamination agent which has a sporicidal effect which causes at least a 3-log reduction; passes in aerosol form into the containment; and may be one of hydrogen peroxide [H₂O₂] and nitrogen dioxide [NO₂] and peroxyacetic acid [C₂H₄O₃] and a mixture of hydrogen peroxide [H₂O₂] and peroxyacetic acid [C₂H₄O₃]; and
the containment is configured as an isolator, lock or restricted access barrier system (RABS), including mobile and stationary kinds, such as a means of transport and rooms for treatment, isolation and/or diagnosis of patients, and also production rooms and laboratories.

5. The catalytic unit as claimed in claim 1, wherein
the catalytic unit is for splitting a decontamination agent present for a decontamination process in a gas stream, passing through the catalytic unit and exiting into an area or a second containment or into the open atmosphere, wherein the exiting gaseous medium has an uncritical concentration of non-degraded decontamination agent amounting to less than 0.5 ppm, whereas a concentration above 1.0 ppm is defined as critical; and
the attained uncritical concentration of non-degraded decontamination agent is not more than 0.1 ppm.

6. The catalytic unit as claimed in claim 1, wherein at least one activated UV light source is directed at the at least one catalytic element during the splitting process to intensify the catalytic effect.

7. The catalytic unit as claimed in claim 6, wherein the at least one UV light source (3) is disposed outside the catalytic unit; or between adjacent catalytic elements; or centrally in a hollow cylindrical catalytic unit comprising one or more catalytic elements.

8. The catalytic unit as claimed in claim 6, wherein one UV light source is arranged in each case outside the catalytic unit; between adjacent catalytic elements; and centrally in a hollow cylindrical catalytic unit comprising one or more catalytic elements.

9. The catalytic unit as claimed in claim 6, wherein to maximize the intensification of the catalytic effect
the at least one UV light source is constituted and positioned and/or guide elements are disposed on the UV light source and/or on the catalytic unit such that a maximum of the radiative effect of the UV light source is attainable on a maximum of the surface area of the one catalytic element or the two or more catalytic elements; and/or
the wavelength of the at least one UV light source is adjusted according to the constitution of the carrier influenced catalytically active component.

10. The catalytic unit as claimed in claim 2, wherein the catalytic unit comprises:
at least one plate-shaped catalytic element or two or more consecutively packed such catalytic elements having a gastight covering possessing an open inlet for inflowing a process stream into the catalytic unit and an open outlet for outflowing the process stream out of the catalytic unit; or
at least one hollow cylindrical catalytic element or two or more concentrically packed such catalytic elements having a gastight covering possessing an open inlet for inflowing the process stream into the catalytic unit and an open outlet for outflowing the process stream out of the catalytic unit.

11. The catalytic unit as claimed in claim 2, wherein
the catalytic unit is for splitting a decontamination agent which has a sporicidal effect which causes at least a 3-log reduction; passes in aerosol form into the containment; and may be one of hydrogen peroxide [H₂O₂] and nitrogen dioxide [NO₂] and peroxyacetic acid [C₂H₄O₃] and a mixture of hydrogen peroxide [H₂O₂] and peroxyacetic acid [C₂H₄O₃]; and
the containment is configured as an isolator, lock or restricted access barrier system (RABS), including mobile and stationary kinds, such as a means of transport and rooms for treatment, isolation and/or diagnosis of patients, and also production rooms and laboratories.

12. The catalytic unit as claimed in claim 3, wherein
the catalytic unit is for splitting a decontamination agent which has a sporicidal effect which causes at least a 3-log reduction; passes in aerosol form into the containment; and may be one of hydrogen peroxide [H₂O₂] and nitrogen dioxide [NO₂] and peroxyacetic acid [C₂H₄O₃] and a mixture of hydrogen peroxide [H₂O₂] and peroxyacetic acid [C₂H₄O₃]; and
the containment is configured as an isolator, lock or restricted access barrier system (RABS), including mobile and stationary kinds, such as a means of transport and rooms for treatment, isolation and/or diagnosis of patients, and also production rooms and laboratories.

13. The catalytic unit as claimed in claim 2, wherein
the catalytic unit is for splitting a decontamination agent present for a decontamination process in a gas stream, passing through the catalytic unit and exiting into an area or a second containment or into the open atmosphere, wherein the exiting gaseous medium has an uncritical concentration of non-degraded decontamination agent amounting to less than 0.5 ppm, whereas a concentration above 1.0 ppm is defined as critical; and
the attained uncritical concentration of non-degraded decontamination agent is not more than 0.1 ppm.

14. The catalytic unit as claimed in claim 3, wherein
the catalytic unit is for splitting a decontamination agent present for a decontamination process in a gas stream, passing through the catalytic unit and exiting into an area or a second containment or into the open atmosphere, wherein the exiting gaseous medium has an uncritical concentration of non-degraded decontamination agent amounting to less than 0.5 ppm, whereas a concentration above 1.0 ppm is defined as critical; and
the attained uncritical concentration of non-degraded decontamination agent is not more than 0.1 ppm.

15. The catalytic unit as claimed in claim 4, wherein
the catalytic unit is for splitting a decontamination agent present for a decontamination process in a gas stream, passing through the catalytic unit and exiting into an area or a second containment or into the open atmosphere, wherein the exiting gaseous medium has an uncritical concentration of non-degraded decontamination agent amounting to less than 0.5 ppm, whereas a concentration above 1.0 ppm is defined as critical; and
the attained uncritical concentration of non-degraded decontamination agent is not more than 0.1 ppm.

16. The catalytic unit as claimed in claim 2, wherein at least one activated UV light source is directed at the at least one catalytic element during the splitting process to intensify the catalytic effect.

17. The catalytic unit as claimed in claim 3, wherein at least one activated UV light source is directed at the at least one catalytic element during the splitting process to intensify the catalytic effect.

18. The catalytic unit as claimed in claim 4, wherein at least one activated UV light source is directed at the at least one catalytic element during the splitting process to intensify the catalytic effect.

19. The catalytic unit as claimed in claim 5, wherein at least one activated UV light source is directed at the at least one catalytic element during the splitting process to intensify the catalytic effect.

20. The catalytic unit as claimed in claim 7, wherein to maximize the intensification of the catalytic effect
the at least one UV light source is constituted and positioned and/or guide elements are disposed on the UV light source and/or on the catalytic unit such that a maximum of the radiative effect of the UV light source is attainable on a maximum of the surface area of the one catalytic element or the two or more catalytic elements; and/or
the wavelength of the at least one UV light source is adjusted according to the constitution of the carrier influenced catalytically active component.

21. The catalytic unit as claimed in claim 8, wherein to maximize the intensification of the catalytic effect
the at least one UV light source is constituted and positioned and/or guide elements are disposed on the UV light source and/or on the catalytic unit such that a maximum of the radiative effect of the UV light source is attainable on a maximum of the surface area of the one catalytic element or the two or more catalytic elements; and/or
the wavelength of the at least one UV light source is adjusted according to the constitution of the carrier influenced catalytically active component.
